(19) United States
(12) Patent Application Publication
(10) Pub. No.: US 2016/0059980 A1
(43) Pub. Date: Mar. 3, 2016

(54) METHODS FOR PACKAGING AND SHIPPING SURGICAL INSTRUMENTS AND IMPLANTS

(71) Applicant: Howmedica Osteonics Corp., Mahwah, NJ (US)

(72) Inventor: Mark Nemec, Goshen, NY (US)

(21) Appl. No.: 14/819,032

(22) Filed: Aug. 5, 2015

Related U.S. Application Data
(60) Provisional application No. 62/041,983, filed on Aug. 26, 2014.

(51) Int. Cl. B65B 55/12; A61F 2/00; A61L 2/08; A61B 19/02

(52) U.S. Cl. CPC B65B 55/12; A61B 19/026; A61F 2/0095; A61L 2/081; A61L 2/082; A61B 2019/0267

(57) ABSTRACT

A method of delivering implants and instruments to a healthcare facility includes receiving at least one container containing used instruments and unused implants. The used instruments and unused implants are processed, sterilized, and placed into respective containers. The containers are shipped to the healthcare facility.

### CROSS-REFERENCE TO RELATED APPLICATIONS

The present application claims the benefit of the filing date of U.S. Provisional Patent Application No. 62/041,983, filed Aug. 26, 2014, the disclosure of which is hereby incorporated herein by reference.

### BACKGROUND OF THE INVENTION

Central Services Departments (“CSD”) are the central hub of healthcare facilities (e.g., hospitals, ambulatory surgery centers and the like), for receiving, processing, and distributing surgical supplies, including surgical instruments and implants used in orthopedic surgeries. In many of these healthcare facilities, numerous surgical procedures are performed each day. As such, large quantities of surgical instruments meant for different procedures and supplied by various sources pass through CSDs on a daily basis, where they may be decontaminated, assembled in sets, packaged, sterilized, stored and distributed when needed.

Some of the general types of surgical instruments that pass through CSDs are reusable, custom/patient specific and disposable instruments, all of which may be utilized in a single procedure. Unused reusable instruments typically arrive at the healthcare facility and are sent to the CSD to be cleaned/decontaminated, sterilized, and packaged for surgery. Once used, such instruments may then enter into a cycle of use where they may pass through a CSD on numerous occasions. Thus, it is common for custom and disposable instruments and implants to arrive separately to a CSD from the source and be processed separately from reusable instruments utilized in the same procedure.

While CSDs have proven to be beneficial since their inception for helping to provide sterile equipment to the operating room, their implementation and use has led to many drawbacks, particularly in an age where the number of surgical procedures and their associated costs are on the rise and where drug resistant microorganisms are frequent residents of hospitals and other healthcare facilities. One such drawback is that, due to the sheer volume and different types of instruments handled on a daily basis, there is an inevitable loss of such instruments each year. This consequently results in the loss of tens of thousands, if not hundreds of thousands, of dollars. Additionally, the separate processing of instruments utilized in the same procedure may result in the wrong instruments ending up in the operating room.

CSDs generally have varying procedures for handling unsterilized equipment particularly when such equipment is delivered from an outside source, which can result in a great deal of discrepancy in their state of sterility when they arrive in the operating room. Further, after surgery, some reusable instruments may be dumped in a tub, sprayed with an cleaning agent, and delivered to the CSD where they may sit for hours or days, which can result in corrosion, sometimes to a point where the instruments must be discarded leading to further monetary losses.

### BRIEF SUMMARY OF THE INVENTION

Disclosed herein are systems, devices and methods for packaging, delivering, and handling surgical instruments and implants. Such systems, devices, and methods, among other things, allow surgical instruments and implants to bypass a healthcare facility's CSD both when the instruments and implants are delivered to an operating room from an outside source and after the surgical procedure has concluded.

Some of the methods disclosed herein may be joined together to form a general cycle of distribution and use of surgical instruments and implants. In such a cycle, a source of the instruments and implants, such as a manufacturer, supplier, distributor, and the like, prepares the instruments and/or implants for delivery to an operating room or operating theater located within a healthcare facility. Such instruments and implants are packaged, sterilized and shipped to the healthcare facility where they are directly delivered to the operating room bypassing the facility's CSD. The sterile instruments and implants are then arranged for surgery. After the surgical procedure has taken place, the used and now contaminated instruments as well as the unused implants are prepped in the operating room for return to the source. Such preparation safeguards the contaminated instruments from transmission of microorganisms until they arrive back to the source and once again bypass the facility's CSD. Once received by the source, such instruments are cleaned, disinfected, supplemented by new instruments (if necessary), repackaged, and sterilized for delivery to the same healthcare facility or another healthcare facility to be used in another surgical procedure. Similarly, any prepackaged unused implants are inspected for damage and contamination by a non-sterile environment, restocked for future use (if clean or undamaged), and/or supplemented by new prepackaged implants (if necessary). Non-packaged, unused implants or otherwise contaminated implants are inspected, replaced (if necessary), sterilized, and prepared for shipment.

In one aspect of the present disclosure, a method of delivering surgical instruments and implants to a healthcare facility includes the steps of obtaining the instruments and implants from an operating room after the completion of a surgical procedure, placing the instruments into at least one instrument container, placing the implants into at least one implant container, sterilizing the instruments and implants, and shipping the at least one instrument container having sterilized instruments therein and at least one implant container having sterilized implants therein to the healthcare facility.

In addition, the method may include the steps of placing the at least one instrument container into a first sterilization bag, and placing the at least one instrument container and first sterilization bag into a second sterilization bag. A further step may include vacuum sealing the at least one instrument container and at least one implant container.

The method may also include the steps of placing the at least one instrument container into a first clean container, and placing the at least one implant container into a second clean container. Also, placing the first and second clean containers into a shipping container may be included in the method. Alternatively, the method may include the steps of placing the first clean container into a first shipping container, and placing the second clean container into a second shipping container. A return kit may be placed into the first clean container. The return kit may include at least one contamination container therein. Sterilizing the implants may include sterilizing the implants while contained within the clean container.

Continuing with this aspect, the instruments may include femoral instruments, tibial instruments, and patellar instruments, and the at least one instrument container may include a first, second and third instrument container. Additionally, the instrument placing step may include placing the femoral instruments into the first instrument container, placing the tibial instruments into the second instrument container, and placing the patellar instruments into the third instrument container.

Alternatively, the instruments may include femoral instruments and acetabular instruments, and the at least one instrument container may include a first and second instrument container. Additionally, the instrument placing step may include placing the femoral instruments into the first instrument container, and placing the acetabular instruments into the second instrument container.

In an additional aspect of the present disclosure, a method of delivering implants and instruments to a healthcare facility includes the steps of receiving at least one container containing used instruments and unused implants, processing the used instruments and unused implants, sterilizing the used instruments and unused implants, placing the used instruments into at least one instrument container, and shipping the at least one instrument container and at least one implant container containing the unused implants to the healthcare facility.

In addition, the processing step may include decontaminating the used instruments, inspecting the used instruments for damage, and inspecting the unused implants for contact with an unsterile environment. Also, the processing step may include inventorying the used instruments, and storing the used instruments in a storage facility. The instruments may include femoral instruments, tibial instruments, and patellar instruments. The method may include the step of removing the used instruments from a contamination container.

In yet another aspect of the present disclosure, a method of delivering implants and instruments to a healthcare facility includes the steps of receiving used instruments previously utilized in a surgical procedure, processing the used instruments, sterilizing the used instruments, and shipping the used instruments to the healthcare facility.

Also, the processing step may include decontaminating the used instruments, and inspecting the used instruments for damage. The method may also include removing the used instruments from a contamination container. In addition, the method may include the steps of receiving a first set of implants from an operating room, and inspecting the first set of implants for contact with an unsterile environment. Additional steps may include manufacturing unused instruments and a second set of implants, sterilizing the unused instruments and the second set of implants, and packaging the used instruments, unused instruments, and first and second sets of implants. The shipping step may include shipping the unused instruments and first and second sets of implants to the healthcare facility.

In another aspect of the present disclosure, a method of handling surgical instruments includes the step of receiving delivery from a source external to a healthcare facility of at least one instrument container containing the instruments therein in a sterile state. The method further includes the step of moving the at least one instrument container while the instruments remain in a sterile state to an operating room located within the healthcare facility. Also included in the method is the step of performing a surgical procedure with the instruments. In addition, the method includes the steps of placing the instruments into the at least one instrument container while located in the operating room, and shipping the instrument container containing the instruments back to the source.

Additionally, the method may include the step of removing the at least one instrument container from a shipping container prior to the moving step. The method may also include the step of removing the at least one instrument container from a clean container after the moving step.

Continuing with this aspect, the method may include the steps of penetrating a first sterile environment in which the instrument container is enclosed, and penetrating a second sterile environment in which the instrument container is enclosed. The method may also include the steps of placing the at least one instrument container into a contamination container, and placing the at least one instrument container and contamination container into a shipping container.

In another aspect of the present invention, a method of handling surgical instruments includes the steps of receiving delivery from a source external to a healthcare facility of at least one instrument container containing the instruments therein in a sterile state. Also included in the method is the step of moving the at least one instrument container while the instruments and implants remain in a sterile state to an operating room located within the healthcare facility. Additionally, the method includes the steps of placing the instruments into the at least one instrument container while located in the operating room after the instruments were used in a surgical procedure, and shipping the instrument container containing the instruments back to the source.

Additionally, the method may include the step of removing the at least one instrument container from a shipping container prior to the moving step. The method may also include the step of removing the at least one instrument container from a clean container after the moving step. Additionally, the method may include the steps of placing the at least one instrument container into a contamination container, and placing the at least one instrument container and contamination container into a shipping container.

In a further aspect of the present invention, a method of handling surgical instruments includes the step of receiving delivery from a source external to a healthcare facility of a first clean container and a second clean container. The first clean container contains surgical instruments, and the second clean container contains implants. The surgical instruments and implants are in a sterile state. The method also includes the step of moving the first and second clean containers to an operating room located within the healthcare facility while the instruments and implants remain in the sterile state. Additionally, the method includes the step of performing a surgical procedure with the instruments and at least one implant. Further, the method includes the steps of placing the instruments into the first clean container and the implants that were not used during the surgical procedure into the second clean container while located in the operating room, and shipping the first and second clean containers containing the instruments and implants that were not used during the procedure back to the source.

Continuing with this aspect, the method may also include the step of placing the instruments used during the surgical procedure into a contamination bag prior to the step of placing the instruments into the first clean container.

### BRIEF DESCRIPTION OF THE DRAWINGS

The features, aspects, and advantages of the present invention will become better understood with regard to the following description, appended claims, and accompanying drawings in which:

FIG. 1 is a flow chart of one embodiment method of delivering surgical instruments and implants to a healthcare facility.

FIG. 2 is a diagram depicting the flow of the surgical instruments and implants in the method of FIG. 1 from receipt at the healthcare facility to their departure from the healthcare facility.

FIG. 3 is a flow chart of one embodiment of a method of handling the implants and instruments delivered pursuant to the method of FIG. 1 from receipt at the healthcare facility to their use in a surgical procedure.

FIG. 4 is a flow chart of one embodiment of a method of handling the implants and instruments delivered pursuant to the method of FIG. 1 from the end of their use in the surgical procedure to their departure from the healthcare facility.

### DETAILED DESCRIPTION

As used herein, the term “sterile” means substantially free from microorganisms, and the term “clean” means non-sterile and generally free from dirt, contaminants and other clinging particulates so as to help minimize transmission of microorganisms. Also, as used herein, the terms “about,” “generally” and “substantially” are intended to mean that slight deviations from absolute are included within the scope of the term so modified.

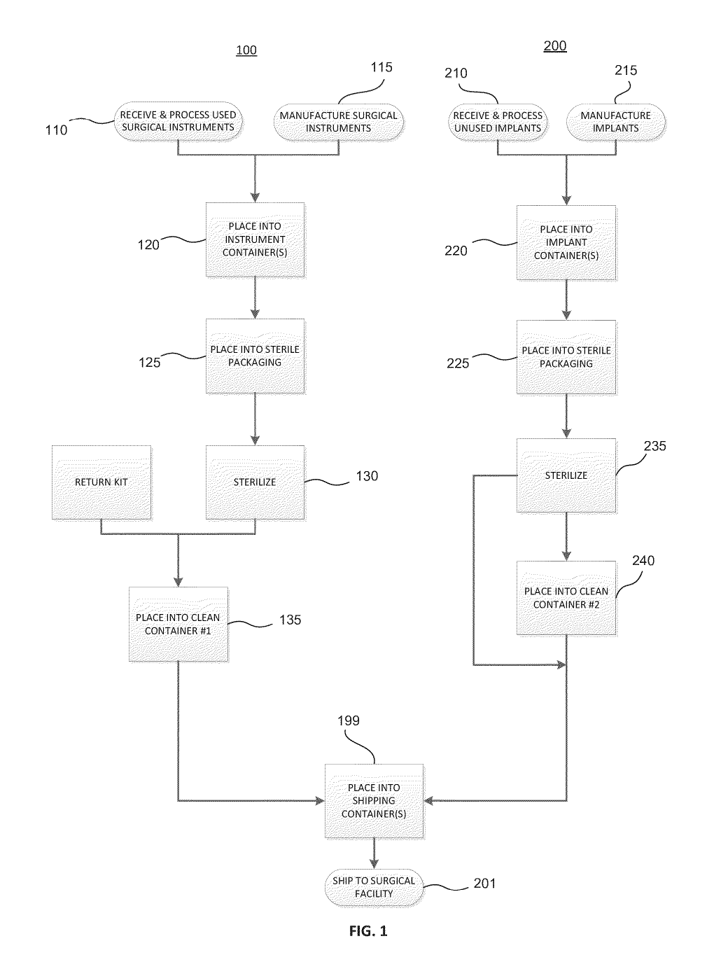

FIG. 1 is a flowchart depicting an embodiment method 100 of preparing surgical instruments for delivery to a healthcare facility. The instruments may include reusable instruments, disposable instruments, and custom instruments, which themselves may be disposable or reusable. The reusable instruments may be newly manufactured or formerly used in a previously performed surgical procedure.

As illustrated, method 100 generally begins by receiving and processing 110 used surgical instruments from a previously performed surgical procedure. As such, most, if not all, of these instruments are contaminated with blood and/or tissue of the patient when they are received by the source.

Once these previously used instruments are received they are processed for packaging. Processing generally includes decontaminating, inspecting, inventorying, and assembling into kits. Decontamination is generally achieved by a physical or chemical process and helps render the previously used instruments safe for further handling. Decontamination may begin by cleaning the instruments, which includes the physical removal of all visible contaminants such as blood and tissue from the instruments' surfaces. Cleaning helps remove barriers which can impede disinfection, which may be the next step in the decontamination process and generally involves a significant removal of microorganisms to render them safe for handling.

Once the previously used instruments are safe to handle, the instruments are inspected to ensure cleanliness and also for damage or wear, in which case they may be discarded if no longer serviceable. The remaining instruments are then inventoried to verify that all the appropriate instruments have been returned from the healthcare facility in order to help reduce the number of instruments lost. Having the source perform this function allows for greater efficiency over that of a CSD, as the source is likely more familiar with their specific instruments and more likely to handle a smaller variety of instruments than a CSD. Additionally, a source may have a larger area to organize instruments and may have more resources to be able to implement a more sophisticated tracking scheme.

During or after the inventorying process, the previously used instruments may be grouped together into a kit comprised of many or all of the instruments needed for a single procedure. Again, having the source perform this function may be beneficial as the source is likely to have expertise in the appropriate grouping of instruments necessary for a particular surgical procedure, and allows these instruments to remain together during the entire cycle of use and distribution, which may further enhance efficient tracking of instruments.

In the event some of the previously used instruments are discarded due to wear, damage, or the like, the source may manufacture 115 new instruments to take the place of the discarded instruments in the kit. These newly manufactured instruments may include reusable, custom/patient specific instruments or disposable instruments to be assembled in a kit alongside the previously used, processed instruments. Thus, in some embodiments the kits may include both previously used and newly manufactured instruments. In other embodiments, the kits may include only previously used instruments. And in further embodiments, the kits may include all newly manufactured instruments. Of course, the initial preparation of a kit may include all newly manufactured instruments.

Once the instruments have been processed, the instruments are packaged 120 into an instrument container, or split amongst multiple instrument containers based on the surgeon's preference or by some other organizational scheme. In one example, where the instruments are prepared for a total knee arthroplasty, the instruments may be divided between four separate instrument containers where all femoral instruments are located in a first container, all tibial instruments are located in a second container, all patellar instruments are located in a third container, and all universal instruments are located in a fourth container. In another example, where the instruments are processed and assembled for a total hip arthroplasty, the instruments may be divided between three instrument containers where all femoral instruments are located in a first container, all acetabular instruments are located in a second container, and all universal instruments are located in a third container.

The instruments may be contained in any commercially available container that allows the instruments to be sterilized while contained therein or sterilized and then placed therein while in a sterile state, as well as allow for such instruments to be protected from contamination during storage and handling before use in a surgical procedure. Some general types of containers that may be utilized include flexible containers, rigid containers, and semi-rigid containers. Flexible containers may be made from flexible material such as textiles, nonwovens, or pouch packaging, for example. A specific example of such flexible containers is disclosed in Int'l Application Publication No. WO 2014/083526, filed Nov. 28, 2013, the disclosure of which is hereby incorporated by reference herein. Rigid containers may include metallic containers or rigid polymeric containers, for example. Semi-rigid containers may include injection molded containers formed from thermoplastic materials that are shaped to include cavities conformed to the specific geometry of each instrument.

Once the instruments are placed within their respective instrument container, the instrument containers are placed 125 into a box and/or sterile packaging, which may include, but is not limited to, sterilization containers, such as sterilization bags, shrink wrap containers, and vacuum containers. Thereafter, the instruments are preferably sterilized 130 while contained within their respective containers. Such sterilization may be achieved through ionizing radiation, which is one of many benefits to the source receiving, processing and sterilizing previously used instruments as ionizing radiation is often not available in healthcare facilities and is commonly understood to be the most effective technique for ensuring complete and uniform sterility. Also, the sterilization of instruments in a packaged kit allows for both previously used instruments and newly manufactured instruments to be sterilized together, which facilitates consistent sterilization between each instrument.

While ionizing radiation is generally preferred, other sterilization techniques may be utilized such as dry heat, nonionizing radiation, gases, plasmas, autoclaving, and chemical solutions, for example. Where such techniques are utilized, the instruments may be sterilized prior to being placed within their respective instrument container. For example, in one embodiment, the instruments and instrument container may be sterilized separately, assembled in a sterile environment, and shrink wrapped, boxed and shrink wrapped or vacuum sealed. In another embodiment, the instruments may be placed within their associated container, sterilized while in the container, for example where the container is open such that the instruments are exposed to the sterilant, and either shrink wrapped, boxed and shrink wrapped or vacuum sealed. In a further embodiment, each instrument may be placed into a sterilization container, such as sterilization bags, and sterilized, such as by autoclaving, and then placed individually into their respective instrument container, which may be separately sterilized.

After the instruments have been sterilized, the instrument containers are placed 135 into further packaging in preparation for shipping. In one embodiment, each sealed and sterilized instrument container, where there are multiple instrument containers, are placed into a first clean container (not shown). In other embodiments, the instrument containers are divided between multiple clean containers. The first clean container is generally any container that maintains clean interior and exterior surfaces to allow the clean container to be carried into an operating room so that the contents may be emptied therein. For example, a clean container can be any commercially available container with a sealable lid, such as a polymer container with a removable or hinged lid that provides an airtight seal.

A return kit may also be placed 135 within the first clean container along with at least one instrument container. A return kit can be any kit that contains items utilized for preparing the instruments after the surgical procedure for their departure from the healthcare facility. In one embodiment, the return kit includes a contamination container or multiple contamination containers so that once the instruments are used, they may be placed within the contamination container for travel back to the source with a reduced risk of transmission of microorganisms. In other embodiments, a return kit may contain other items such as cloths, sponges and/or other soft barriers that may be wrapped or folded around each instrument or a group of instruments to help prevent damage during transport. In one example, a sponge may be placed over three or more instruments, all of which may then be encapsulated in a soft barrier wrapped or tri-folded thereabout. Other items may include wipes and/or cleaning/disinfecting agents, such as enzymatic cleaners, to wipe down the instruments while still in a moist state to help make the cleaning/disinfection process easier once returned to the source.

FIG. 1 also depicts a method embodiment 200 for preparing surgical implants for delivery to a healthcare facility. In many surgical procedures, the surgeon has multiple implants within the operating room from which he or she can select the most appropriate implant or implants for the specific patient. However, the remaining, unselected implants go unused. In one example of a total knee arthroplasty, several implants of varying sizes or types are located within an operating room to give the healthcare professional multiple options to best suit the patient's specific anatomy as determined during the surgical procedure. The remaining implants are left unused. As such, and as depicted in FIG. 1, these remaining, unused implants may be sent back to the source where they are received and processed 210.

Once the unused implants are received, they are processed for packaging. Processing generally includes inspecting, decontaminating, inventorying, and, optionally, assembling in kits. Often, the surgeon or other healthcare professional may not open whatever container the implants are contained in until it is determined which implant is most suitable. Thus, in some embodiments the unused implants may arrive back to the source unopened and still in a sterile state. Inspection verifies whether the implant has been exposed to bodily fluid or an otherwise unsterile environment and whether decontamination is necessary. Inspection of the implant is also utilized to verify whether the implant has suffered any damaged.

Where decontamination is warranted, such decontamination is generally achieved in the same manner as with the surgical instruments as previously described. Thereafter, the implants are inventoried to help ensure accountability of all implants to safeguard against loss. Additionally, new implants may be manufactured 215 by the source to fill in for those implants that had already been implanted in a patient. The new and unused implants may then be assembled into a kit comprised of all of the variety of implants that are desirable to have available during the surgical procedure. In some embodiments, the kit may be comprised of all new implants, such as when initially preparing a kit.

Once the implants are processed, each implant is packaged 220 into one implant container. However, in some embodiments, such implants may be split amongst multiple implant containers. In one example, where the implants are provided for a total knee arthroplasty, the implants may be divided between three separate implant containers where all femoral implants are located in a first container, all tibial implants are located in a second container, and all patellar implants are located in a third container. In another embodiment, the implants may be separated by size. For example, all femoral, tibial, and patellar implants of a first size may be placed in a first container and all femoral, tibial, and patellar implants of a second size may be placed in a second implant container.

The implant containers can be any commercially available container that allows the implants to be sterilized while contained therein or sterilized and then placed therein while in a sterile state, as well as allow for such implants to be protected from contamination during storage and handling before use in a surgical procedure. Some general types of containers that may be utilized include flexible containers, rigid containers, and semi-rigid containers, such as those previously described. One example of an implant container is disclosed in U.S. application Ser. No. 12/655,369, filed Dec. 29, 2009 and hereby incorporated by reference herein as if fully set forth herein.

Once the implants are placed within the implant container, the implant container is then placed into sterile packaging 225 such as a vacuum container, shrink wrap container or a sterilization container, as described above. In one embodiment, the instrument container can also be a vacuum container capable of being vacuum sealed. Similar to the sterile packaging of the instruments, the implant container may be packaged in multiple layers of sterile packaging to form several layers of sterile environments. In one example, an implant container may be placed in a first sterilization container and then placed in a second sterilization container. In another example, an implant container may be placed in a sterilization container and then placed in a shrink wrap or vacuum container. In a further example, an implant container may be placed in a vacuum container and then placed in a sterilization container. In yet another example, an implant container may be placed within a first sterilization container, a second sterilization container and a shrink wrap or vacuum container. It is also contemplated that the implants may be placed into sterile packaging prior to being placed into the implant containers where the sterilization technique so requires.

Thereafter, the implants are sterilized 235 by irradiation or some other technique, as previously described. The sterilized implants and implant containers are preferably placed 240 into a second clean container separate from the first clean container that contains the instruments and return kit. In some embodiments, the implants, instruments and return kit may be placed into the same clean container. In other embodiments, the second clean container can constitute sterile packaging such that the implant containers are placed into the clean container prior to sterilization. In this embodiment, the interior of the second clean container would remain sterile during transport. In some embodiments, the return kit may be placed within the second clean container.

Once the instrument containers and implant containers are placed into their respective clean containers, the clean containers are then placed 199 into a shipping container to keep the instruments and implants together and help the clean containers maintain clean outer surfaces. In some embodiments, the clean containers may couple together. In other embodiments, the clean containers may each be placed into their own respective shipping container and these shipping containers may be coupled together or left separate. The shipping container is then shipped 201 to the healthcare facility.

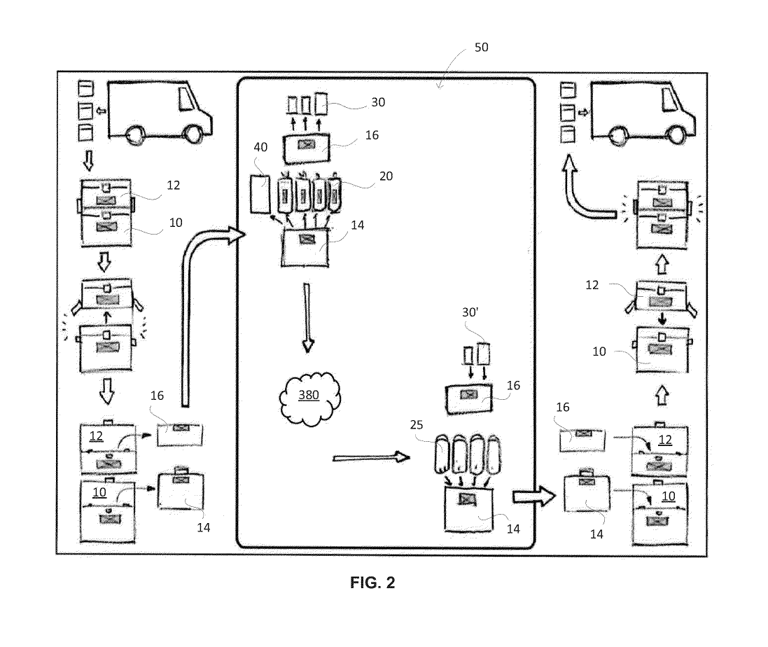

FIG. 2 is a diagrammatic representation of the flow of instruments and implants from arrival to the healthcare facility to departure. As described in further detail below, the instruments and implants (not shown) arrive to the healthcare facility in shipping containers 10 and 12, which may be coupled together. Shipping containers 10 and 12 are separated and a first clean container 14 containing the instruments is removed from shipping container 10, and a second clean container 16 containing the implants is removed from shipping container 12. Clean containers 14 and 16 are moved into the operating room 50 where instrument containers are removed from first clean container 14, and implant containers 30 are removed from second clean container 16. The implants are laid out for surgery according to the surgeon's preference. Each instrument container 20 is removed from a box and/or any other sterilization packaging. The instruments are laid out for surgery 380.

After the surgery 380 is performed, the contents of the return kit 40 are applied and the instruments are returned to instrument containers 20. Each container 20 is then placed into a contamination container 25. Thereafter, contamination containers 25 containing instrument containers 20 therein are placed back into first clean container 14, and the unused implants 30′ are placed back into second clean container 16. Clean containers 14 and 16 are removed from operating room 50 and placed into respective shipping containers 10 and 12, which may be coupled together and are then shipped back to the source.

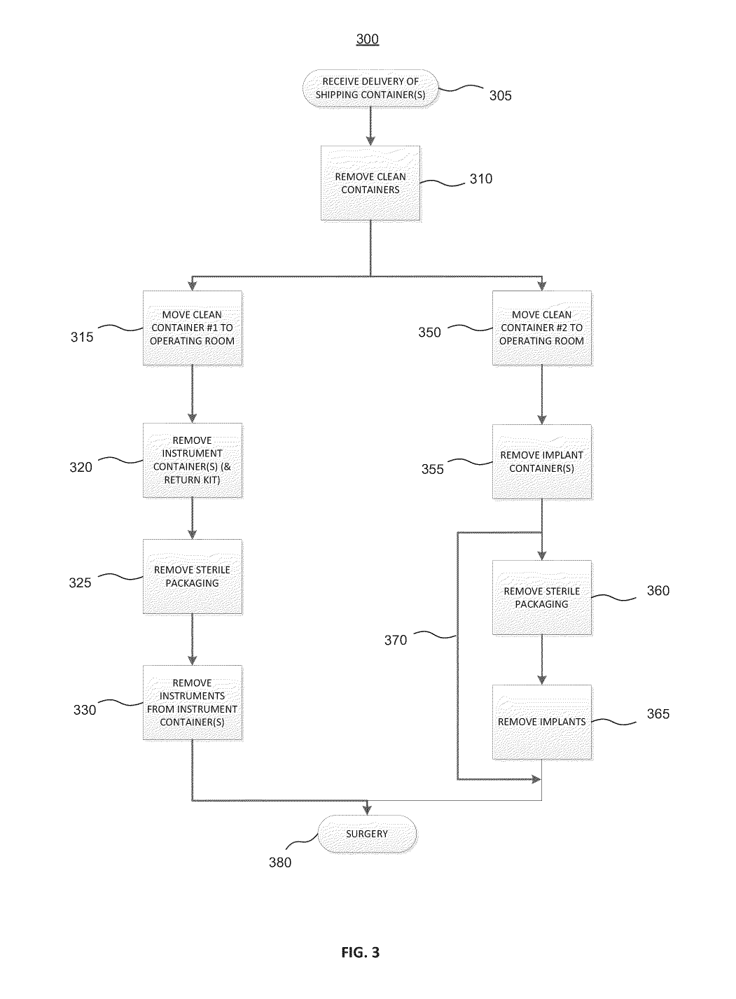

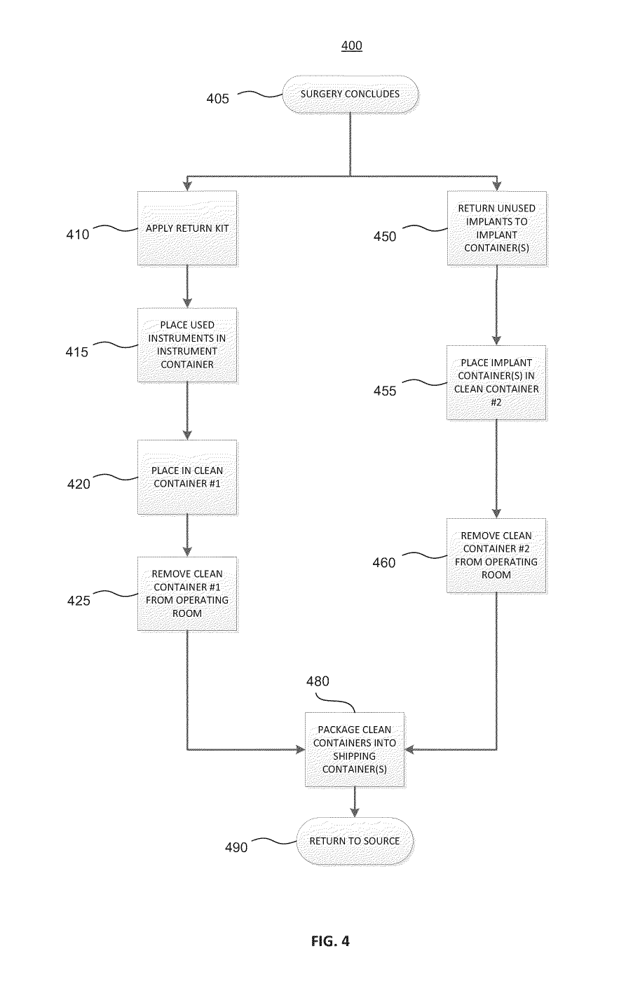

FIGS. 3 and 4 are flow charts corresponding to the diagram of FIG. 2. FIG. 3 illustrates a method embodiment 300 of handling surgical instruments and implants from arrival 305 to the healthcare facility up until the surgical procedure 380. As depicted and as generally described above, once shipping containers 10 and 12 arrive at the healthcare facility, they are received 305 and taken to operating room 50. Prior to entering operating room 50 or the operation wing of the healthcare facility, clean containers 14 and 16 are removed 310 from shipping containers 10 and 12, and then taken into operating room 50.

In some embodiments, a single shipping container containing clean containers 14 and 16 may be received at the healthcare facility. In other embodiments, shipping containers 10 and 12 may be taken into operating room 50 or may be stored in a storage room until operating room 50 is ready to be prepared for the procedure.

Once clean container 14 is moved 315 into operating room 50, instrument containers 20 and, optionally, return kit 40 are removed 320. The sterile layers surrounding instrument containers 20, or around the instruments themselves, are removed 325 and the sterile instruments are arranged for surgery.

In one embodiment, return kit 40 and instrument containers 20 are removed from clean container 14 prior to entering operating room 50. In another embodiment, where each instrument container 20 is contained within multiple layers of sterile packaging, a non-sterile healthcare professional, such as a circulating nurse, may remove each instrument container 20 from clean container 14 and break a first sterile barrier, such as by opening a shrink wrap, sterilization or vacuum container. A sterile healthcare professional, such as a scrub nurse, may then reach through the sterile barrier and remove the contents therein. The sterile healthcare professional may then break a second sterile barrier, such as by opening a shrink wrap, sterilization or vacuum container, and remove a sterile instrument container 20 disposed therein.

With regards to the implants, once clean container 16 is moved 350 into operating room 50, implant containers 30 are removed 355 therefrom. Thereafter, the sterile layers surrounding implant containers 30, or around the implants themselves, are optionally removed 360 and the sterile implants are optionally removed 365 from implant container 30 and arranged for surgery. Where multiple implant containers 30 are provided, it is preferable to leave the implants in their respective implant containers until a particular implant is selected for implantation, which may simplify processing once the unused implants are return to the source.

FIG. 4 illustrates a method embodiment 400 of handling surgical instruments and implants from the conclusion of the surgical procedure 405 to departure 490 from the healthcare facility. As depicted, once the surgical procedure has concluded 405, the used surgical instruments are placed 410 back into instrument containers 20.

The contents of the return kit are removed and the instruments are wiped down or otherwise treated with a cleaning/disinfecting agent and/or wrapped with protective fabric or cushioning material. The cleaning/disinfecting agent helps prepare the instruments for processing once returned to the source. In one embodiment, cleaning wipes may be removed from return kit and used to wipe down the surgical instruments to remove visible blood and tissue in a preliminary cleaning process before decontamination at the source. In other embodiments, the instruments may be placed back into container 20 without being treated with a decontaminant.

Once container 20 is closed, container 20 is placed into at least one contamination container 25. Alternatively, the instruments may be placed into contamination container 25 prior to being placed into container 1020

Instrument containers 20 containing used instruments are then placed 420 back into clean container 14 while located in operating room 50. Clean container 14 helps provide an additional microbial barrier. Thereafter, clean container 14 is removed from operating room 50.

Also after the surgical procedure is concluded 405, the unused implants are placed 450 back into implant containers 30. Implant containers 30′ containing the unused implants are then placed 455 into clean container 16 while located in operating room 50. Thereafter, clean container 16 is removed 460 from operating room 50.

In some embodiments, where the unused implants contacted blood or tissue or were otherwise contaminated, such unused implants may also be placed into a contamination container, such as contamination container 25, either before or after being placed back into implant container 30. In other embodiments, where the implants are contained in multiple implant containers 30 and not removed therefrom during the surgical procedure, implant containers 30′ containing the unused implants may be placed directly into clean container 16.

Clean containers 14 and 16 are then placed 480 back into shipping containers 40 and 12, respectively, or into a single shipping container when only one is provided.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of delivering surgical instruments and implants to a healthcare facility, comprising the steps of:
obtaining the instruments and implants from an operating room after the completion of a surgical procedure;
placing the instruments into at least one instrument container;
placing the implants into at least one implant container;
sterilizing the instruments and implants; and
shipping the at least one instrument container having sterilized instruments therein and at least one implant container having sterilized implants therein to the healthcare facility.

2. The method of claim 1, further comprising the steps of:
placing the at least one instrument container into a first sterilization bag; and
placing the at least one instrument container and first sterilization bag into a second sterilization bag.

3. The method of claim 1, further comprising the steps of:
placing the at least one instrument container into a first clean container; and
placing the at least one implant container into a second clean container.

4. The method of claim 3, further comprising the step of:
placing the first and second clean containers into a shipping container.

5. The method of claim 3, further comprising the steps of:
placing the first clean container into a first shipping container; and
placing the second clean container into a second shipping container.

6. The method of claim 3, further comprising the step of:
placing a return kit into the first clean container, the return kit including at least one contamination container therein.

7. The method of claim 3, wherein the sterilizing step includes:
sterilizing the implants while contained within the clean container.

8. The method of claim 1, wherein the instruments include femoral instruments, tibial instruments, and patellar instruments, and the at least one instrument container includes a first, second and third instrument container.

9. The method of claim 8, wherein the instrument placing step includes:
placing the femoral instruments into the first instrument container;
placing the tibial instruments into the second instrument container; and
placing the patellar instruments into the third instrument container.

10. The method of claim 1, wherein the instruments include femoral instruments and acetabular instruments, and the at least one instrument container includes a first and second instrument container.

11. The method of claim 10, wherein the instrument placing step includes:
placing the femoral instruments into the first instrument container; and
placing the acetabular instruments into the second instrument container.

12. A method of delivering implants and instruments to a healthcare facility comprising the steps of:
receiving at least one container containing used instruments and unused implants;
processing the used instruments and unused implants;
sterilizing the used instruments and unused implants;
placing the used instruments into at least one instrument container; and
shipping the at least one instrument container and at least one implant container containing the unused implants to the healthcare facility.

13. The method of claim 12, wherein the processing step includes:
decontaminating the used instruments;
inspecting the used instruments for damage; and
inspecting the unused implants for contact with an unsterile environment.

14. The method of claim 12, wherein the instruments include femoral instruments, tibial instruments, and patellar instruments.

15. The method of claim 12, further comprising the step of:
removing the used instruments from a contamination container.

16. A method of delivering implants and instruments to a healthcare facility, comprising the steps of:
receiving used instruments previously utilized in a surgical procedure;
processing the used instruments;
sterilizing the used instruments; and
shipping the used instruments to the healthcare facility.

17. The method of claim 16, wherein the processing step includes:
decontaminating the used instruments, and
inspecting the used instruments for damage.

18. The method of claim 16, further comprising the steps of:
receiving a first set of implants from an operating room, and
inspecting the first set of implants for contact with an unsterile environment.

19. The method of claim 18, further comprising the steps of:
manufacturing unused instruments and a second set of implants;
sterilizing the unused instruments and the second set of implants;
packaging the used instruments, unused instruments, and first and second sets of implants.

20. The method of claim 19, wherein the shipping step includes:
shipping the unused instruments and first and second sets of implants to the healthcare facility.
